(19) United States
(12) Patent Application Publication
(10) Pub. No.: US 2012/0165541 A1
(43) Pub. Date: Jun. 28, 2012

(54) METHOD FOR THE PREPARATION OF W-AMINO- ALKANEAMIDES AND W-AMINO-ALKANETHIOAMIDES AS WELL AS INTERMEDIATES OF THIS METHOD

(75) Inventors: Martin Albert, Kundl (AT); Dominic De Souza, Kundl (AT)

(73) Assignee: Sandoz AG

(21) Appl. No.: 13/388,249

(22) PCT Filed: Jul. 30, 2010
(86) PCT No.: PCT/EP2010/004685

§ 371 Date: Mar. 14, 2012

(30) Foreign Application Priority Data
Jul. 31, 2009 (EP) 09167001.8

(51) Int. Cl. C07C 231/14; C07C 237/04; C07D 277/74

(52) U.S. Cl. 548/170; 564/198; 564/197

(57) ABSTRACT

The present invention relates to method for the preparation of an ω-amino-alkane(thio)amide having the general formula (6)

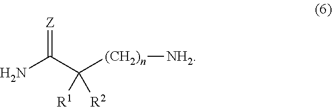

Furthermore, novel intermediates and partial reaction steps of the claimed method are disclosed.

### FIELD OF THE INVENTION

The present invention relates to a method for the preparation of ω-amino-alkaneamides and ω-amino-alkanethioamides. Certain intermediates and partial reaction steps of the method are also claimed. The ω-amino-alkaneamides and ω-amino-alkanethioamides, in particular 3-amino-2,2-dimethylpropionamide, are of particular use in the synthesis of pharmaceuticals such as aliskiren. Aliskiren is a renin inhibitor, which can be used for the treatment of high blood pressure.

### BACKGROUND OF THE INVENTION

3-Amino-2,2-dimethylpropionamide (compound (III), scheme 1) was first disclosed in Buckley; G. D.; Heath; R. L.; Rose; J. D. J. Chem. Soc. 1947, 1500-1504. The process makes use of a conjugate addition of cyanide to nitroalkene (I). The resulting nitroalkane (II) is further reduced to compound (Ill) using Pd as a catalyst in the presence of H₂. The starting material (I) is accessed in a three step sequence from acetone and nitromethane.

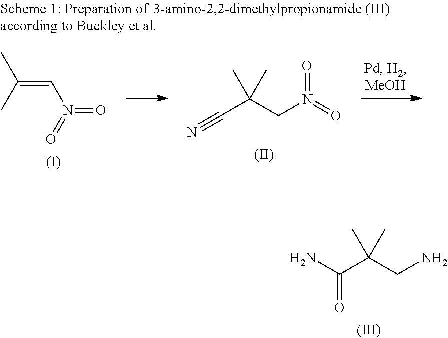

A shorter process is described in WO 2007/071626. Herein, the reduction of a cyanoalkane acid ester such as (IV) using high pressure hydrogenation is disclosed. The conditions required for this conversion are rather harsh thereby limiting the broad applicability of this process. A similar approach using Rh/Al₂O₃ as a catalyst has been disclosed in WO 2006/013094. Here the formation of the amide occurs prior to the reduction of the nitrile.

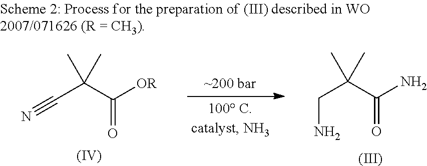

Another variation of this process has been published in Maibaum J., J. Med. Chem. 2007, 50, 4832-4844. Compound (IV) (R═CH₂CH₃) serves as a starting material. The reduction of the nitrile is performed with H₂ using Raney nickel as a catalyst. The amino group is protected prior to the formation of the amide. The amide formation is slow (300 hours) and gives compound (VI) in moderate yield (53% yield). Hydrogenolytic cleavage of the benzyloxycarbonyl (Cbz)-group gives compound (III) (scheme 3).

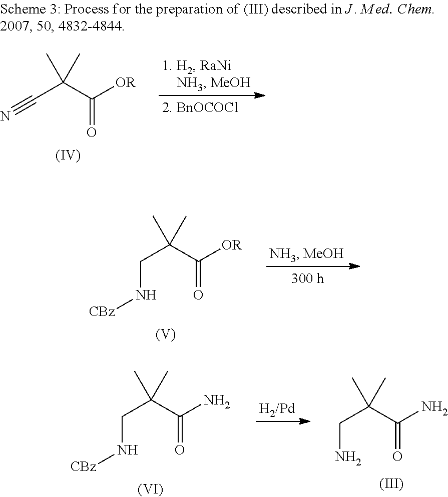

In a similar process (CN 1990461A; see scheme 4) 3-amino-2,2-dimethyl-propionamide (III) is prepared by hydrogenation of cyanodimethylacetic acid amide (VIII), which in turn is accessed by the alkylation of cyanoacetic acid amide (VII). Again, high pressure hydrogenation has to be applied to reduce the cyano group to the aminomethyl group. Additionally, the synthesis of compound (VIII) requires a costly alkylation of cyanoacetic acid amide.

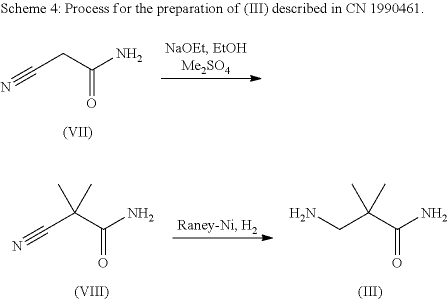

A variation of this process using protecting groups has been published by Dong, H. et al. in Tetrahedron Lett. 2005, 46, 6337-6340.

A different approach for the synthesis of compound (III) avoiding any kind of hydrogenation has been disclosed in AT 502 804 (scheme 5). Though the process does not require a hydrogenation step, the conversion of (XI) to (III) has to be run in high pressure equipment in order to achieve reasonable reaction times. The overall yield of the desired product is rather low as the last two steps (from (X) to (XI) and (XI) to (III)) have a yield of less than 50% each resulting in an overall yield from (IX) to (III) of less than 25%.

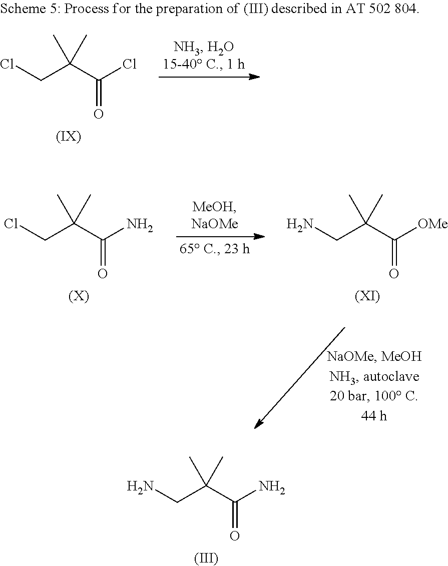

In EP-A-1 548 024 (scheme 6) a process is disclosed which uses an elaborate protecting group strategy combined with an oxidation to give compound (III). Due to the length of the synthetic sequence and the use of costly catalysts the process is not suitable for commercial production of compound (III).

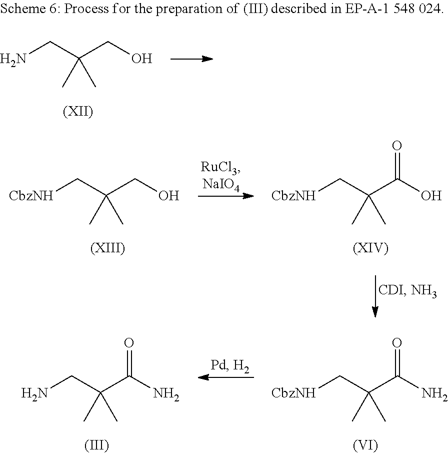

The reaction of compound (XV), obtained by alcoholysis of acid chloride (IX), with hydrazine yielding compound (XVI) (after ring closure) has been described in J. Am. Chem. Soc. 2003, 125, 10778-10779. However, due to the low reactivity of (XV) towards hydrazine, the reaction was slow (43 h at 120° C.) and gave product (XVI) in low yield (30%).

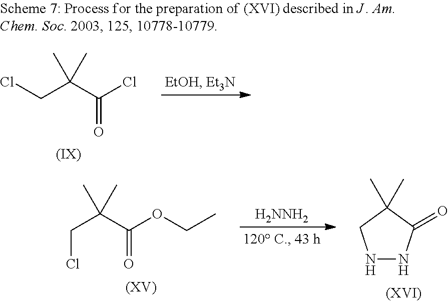

In view of the above, it was an object of the present invention to provide a method for the preparation of ω-amino-alkaneamides and ω-amino-alkanethioamides having the general formula (6) and in particular 3-amino-2,2-dimethylpropionamide having the formula (12) which can provide the product in a good yield. Furthermore, the method should be amenable for production on an industrial scale. In the following the ω-amino-alkaneamide and ω-amino-alkanethioamide are generally referred to as ω-amino-alkane(thio)amide.

### SUMMARY OF THE INVENTION

In one embodiment the present invention relates to a method for the preparation of an ω-amino-alkane(thio)amide (6), wherein the method comprises the steps of:
(a) reacting a compound having the general formula (1) with a compound having the general formula (2) to form a compound having the general formula (3)

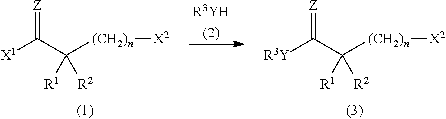

wherein
X¹ is selected from the group consisting of halogen and R—C(O)—O—, wherein R is a C₁₋₈ alkyl group;
X² is a leaving group;
Z is O or S;
R¹ and R² are independently selected from the group consisting of H and C₁₋₆ alkyl, wherein at most one of R¹ and R² is H;
n is an integer from 1 to 5;
R³ is selected such that the compound having the general formula (2) has a pKa value of at most about 11; and
Y is selected from the group consisting of O, NH and S;
(b) subsequently reacting the compound having the general formula (3) with hydrazine to form a compound having the general formula (4)

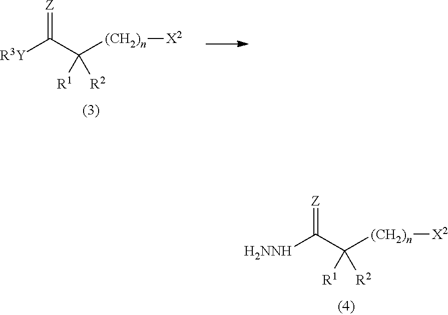

(c) cyclization of the compound having the general formula (4) to form a compound having the general formula (5)

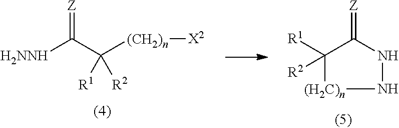

and
(d) opening the ring of the compound having the general formula (5) to form the ω-amino-alkane(thio)amide of the general formula (6)

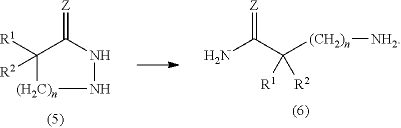

Specifically the invention relates to a method for the preparation of 3-amino-2,2-dimethylpropionamide having the formula (12), the method comprising the steps of:
(a) reacting a compound having the formula (7) with a compound having the formula (8) to form a compound having the formula (9)

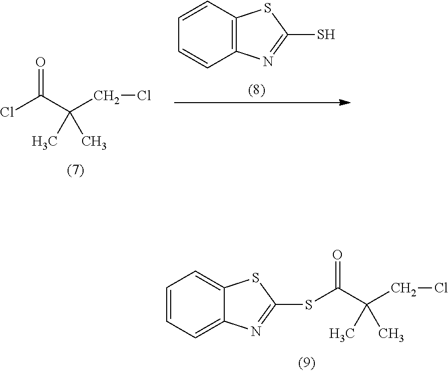

(b) subsequently reacting the compound having the formula (9) with hydrazine to form a compound having the formula (10)

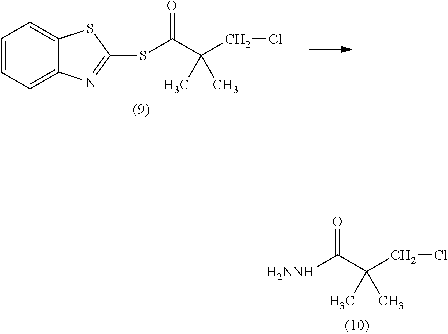

(c) cyclisation of the compound having the formula (10) to form a compound having the formula (11)

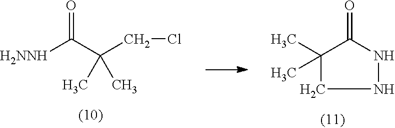

and
(d) opening the ring of the compound having the formula (11) to form 3-amino-2,2-dimethylpropionamide having the formula (12)

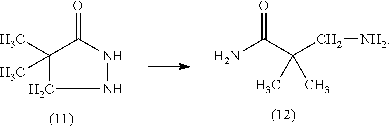

In yet another embodiment the invention refers to a method for the preparation of a compound having the general formula (4a), the method comprising the steps of:
(a) reacting a compound having the general formula (1 a) with a compound having the general formula (2) to form a compound having the general formula (3a)

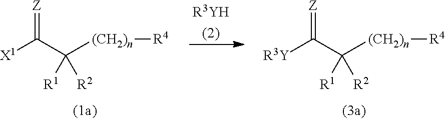

wherein
X¹ is selected from the group consisting of halogen and R—C(O)—O—, wherein R is a C₁₋₈ alkyl group;
Z is O or S;
R¹ and R² are independently selected from the group consisting of H and C₁₋₈ alkyl, wherein at most one of R¹ and R² is H;
n is an integer from 1 to 5;
R⁴ is selected from the group consisting of hydrogen, optionally substituted alkyl, optionally substituted aryl, optionally substituted alkaryl, optionally substituted alkenyl, optionally substituted alkinyl, optionally substituted heteroaryl, optionally substituted heterocyclyl, and a leaving group;
R³ is selected such that the compound having the general formula (2) has a pKa value of at most about 11; and
Y is selected from the group consisting of O, NH and S; and
(b) subsequently reacting the compound having the general formula (3a) with hydrazine to form a compound having the general formula (4a)

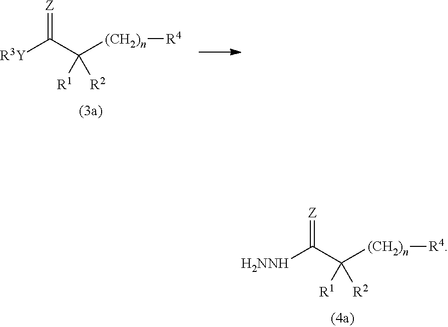

In another embodiment the present invention relates to a compound having the general formula (13a)

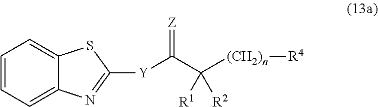

wherein Y, Z, R¹, R² and n have the same meanings as given above and R⁴has the same meaning as X² above and is a leaving group.

In a further embodiment the invention pertains to a compound having the general formula (4a)

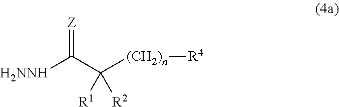

wherein Z, R¹, R², and n have the same meanings as given above and R⁴ has the same meaning as X² above and is a leaving group.

A further embodiment of the invention pertains to the use of a compound having the general formula (2) for the preparation of a compound having the general formula (4a) starting from a compound having the general formula (1a), wherein the compounds having the general formulae (1a), (2), and (4a) are as defined above. In this reaction preferably at most 15 mol-% diacylated compound having the general formula (15a)

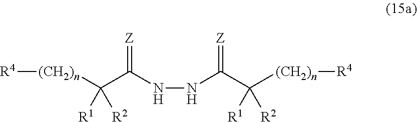

is formed; this can alternatively be expressed as a molar ratio of the compound of the general formula (4a) to the compound (15a) of at least 6,66.

DEFINITIONS

Unless defined otherwise, the following definitions apply within the context of the present invention. It is self-evident that the specific compounds which are employed in the individual reactions steps must permit the desired reaction step to proceed and should not interfere with the desired reaction or lead to undesired by-products.

An “alkyl group” preferably refers to a C₁₋₈ alkyl group, more preferably to a C₁₋₄ alkyl group. Examples of suitable alkyl groups include methyl, ethyl, isopropyl, butyl and tert.-butyl. The alkyl group can be straight, branched or cyclic.

An “alkenyl group” preferably refers to a C₁₋₈ hydrocarbon group which includes at least one double bond, more preferably to a C₁₋₄ alkenyl group.

An “alkinyl group” preferably refers to a C₁₋₈ hydrocarbon group which includes at least one triple bond, more preferably to a C₁₋₄alkinyl group.

An “aryl group” preferably refers to a C₅₋₁₂ aryl group, more preferably to a C₆₋₁₀ aryl group. Examples of suitable aryl groups include phenyl and naphthyl.

A “heteroaryl group” preferably refers to a heteroaryl group containing a five- to twelve-membered ring and having at least one heteroatom selected from N, S and O, more preferably to a heteroaryl group containing a six- to ten-membered ring and having at least one heteroatom selected from N, S and O. Examples of suitable heteroaryl groups include pyrrol, imidazole, triazole, pyridine, furane, thiophene, oxazole, and thiazole. Also included are derivatives thereof in which the heteroaryl ring is anellated to a phenyl ring.

A “heterocyclyl group” preferably refers to a heterocyclic group containing a five- to twelve-membered ring and having at least one heteroatom selected from N, S and O, more preferably to a heterocyclic group containing a six- to ten-membered ring and having at least one heteroatom selected from N, S and O. Examples of suitable heterocyclic groups include pyrrolidine, tetrahydrofuran, tetrahydrothiophene, imidazolidine, piperidine, tetrahydropyran, and piperazine.

An “arylalkyl group” refers to a group in which an aryl group as defined above is covalently bound to an alkyl group as defined above.

A “heteroarylalkyl group” refers to a group in which a heteroaryl group as defined above is covalently bound to an alkyl group as defined above.

An “alkaryl group” refers to a group in which an alkyl group as defined above is covalently bound to an aryl group as defined above.

An “acyl group” is defined as —C(O)—.

A “(hetero)arylacyl group” refers to a group in which a (hetero)aryl group as defined above is covalently bound to an acyl group as defined above.

A “leaving group” refers to a chemical moiety which, under suitable reaction conditions, departs from the compound with a pair of electrons in a heterolytic bond cleavage.

Preferably, the leaving group, after departing, is a neutral or an anionic moiety, more preferably an anionic moiety.

The above mentioned groups can be substituted or unsubstituted by one or more substituents. Examples of possible substituents include -halogen, —CHal₃, —CN, —NC, —NR₂ (wherein R is H or C₁ alkyl), —NO₂, -alkyl, —C(O)-alkyl, —C(S)-alkyl, -aryl, —C(O)-aryl and —C(S)-aryl.

In the absence of a divergent definition commonplace chemical terms as used herein have the meaning that the skilled person ascribes to them, in particular as defined in the chemical dictionary Römpp online, Version 3.5, incorporated herein by reference.

### DETAILED DESCRIPTION OF THE INVENTION

The present invention relates to a method for the preparation of an ω-amino-alkane(thio)amide (6). In a preferred embodiment the ω-amino-alkane(thio)amide is 3-amino-2,2-dimethylpropionamide.

Step (a):

In step (a) a compound having the general formula (1) is reacted with a compound having the general formula (2) to form a compound having the general formula (3).

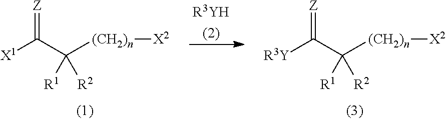

In the general formula (1) X¹ is selected from the group consisting of halogen and R—C(O)—O—, wherein R is a C₁₋₈ alkyl group, preferably a C₁₋₄ alkyl group. Preferably X¹ is halogen, more preferably chlorine.

X² is a leaving group. The type of leaving group is not particularly limited but is preferably selected from the group consisting of halogen; —OSO₂R, wherein R is a C₁₋₄ alkyl group which is optionally substituted with one or more halogens (e.g., mesylate or triflate) or wherein R is a C₅₋₁₂ aryl which is optionally substituted with C₁₋₄ alkyl, NO₂ or CN (e.g., tosylate). Preferably X² is halogen, more preferably X² is chlorine.

Z can be O or S and is preferably O.

R¹ and R² are independently selected from the group consisting of H and C₁₋₈ alkyl, wherein at most one of R¹ and R² is H. Preferably R¹ and R² are C₁ alkyl, more preferably R¹ and R² are methyl.

n is an integer from 1 to 5. In a preferred embodiment n is 1 or 2 and in a more preferred embodiment n is 1.

The starting material (1) of the method of the present invention is commercially available or can be prepared by standard procedures which are known in the art. A preferred starting material is chloropivalic acid chloride (7).

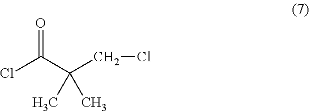

In the compound having the general formula (2), R³YH, Y is selected from the group consisting of O, NH and S, preferably Y is S or O, more preferably S.

R³ is selected such that the compound having the general formula (2) has a pKa value of at most about 11, preferably at most about 10, like at most about 9 or even at most about 8. In a preferred embodiment the pKa of compound (2) is at least 0. In further embodiments the pKa value is from about 1 to about 10, like from about 2 to about 9, from about 3 to about 9, from about 2 to about 8.5, from about 3 to about 8.5 or from about 4 to about 8.5. The pKa value is determined according to the method described in the “Experimental Section” of Boraei, A. A. A. et al., J. Chem. Eng. Data, 1996, 41 (4), 787-790. The pKa is determined using the pure compound having the general formula (2) (assay ≧99 weight-%). The measurement is conducted using a mixture of DMSO and water (molar fraction of DMSO 0.30) at 25° C. and an ionic strength of I=0.02 mol dm⁻³ (KNO₃). All solvents and solutions used for the determination of the pKa value have to comply with the quality described in the “Material and Solutions”-section in Boraei, A. A. A. et al. J. Chem. Eng. Data, 1996, 41 (4), 787-790 and have to be treated and prepared as described therein. The titrations and calculations of the pKa values have to be performed using the procedure, instruments and equations described in the “Procedure”-section in Boraei, A. A. A. et al. J. Chem. Eng. Data, 1996, 41 (4), 787-790 using the average value of three measurements and—for polyacids—the pKa value for the acid—base pair: neutral compound—conjugated base having one negative charge.

R³ can be any group which results in a compound having the general formula (2) with the recited pKa value and does not possess groups which result in side reactions when it is reacted with the compound having the general formula (1). Generally R³ will be an electron withdrawing group. In one embodiment examples of suitable groups include substituted or unsubstituted aryl groups, substituted or unsubstituted heteroaryl groups, substituted or unsubstituted arylalkyl groups, substituted or unsubstituted heteroarylalkyl groups, substituted or unsubstituted arylacyl groups, and substituted or unsubstituted heteroarylacyl groups.

In a preferred embodiment R³ is selected from

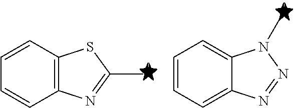

More preferably R³ is

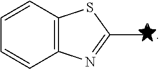

In a further embodiment, the compound having the general formula (2) can be a derivative of an inorganic or organic acid which has a pKa value in the required range. Examples of derivatives include esters, amides and thioamides (e.g., alkyl esters, alkyl amides, alkyl thioamides). Examples of suitable acids include carboxylic acid, phosphoric acid, phosphonic acid, thiophosphoric acid, sulfuric acid and sulfonic acid. Preferred examples include

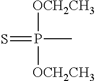

Examples of the compound having the general formula (2) include:

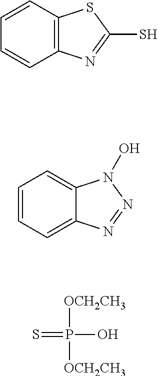

A preferred compound having the general formula (2) R³YH is mercaptobenzothiazol (8).

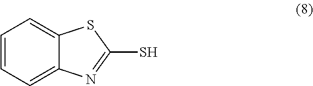

The reaction conditions for step (a) are not particularly limited as long as the compounds of the general formulae (1) and (2) are capable of reacting with each other. If the by-product H—X¹ is acidic, the reaction is preferably performed in the presence of base in order to neutralize this by-product. Examples of suitable inorganic bases include but are not limited to ammonium, alkali or alkaline earth hydroxides (e.g., NH₄OH, NaOH, KOH, LiOH) or ammonium, alkali or alkaline earth carbonates (e.g., Na₂CO₃, K₂CO₃, or Li₂CO₃), or ammonium, alkali or alkaline earth hydrogencarbonates (e.g., NaHCO₃, KHCO₃, or LiHCO₃). Examples of suitable organic bases include but are not limited to tertiary amines such as trialkylamines (e.g., tri(C₁₋₄alkyl)amines such as triethylamine), Hünig's base, amidine and guanidine bases like 1,8-diazabicyclo[5.4.0]undec-7-ene, and aromatic nitrogen-containing heterocycles such as pyridine, 4-(dimethylamino)pyridine, azole, and imidazole. The employed base is preferably an alkali or alkaline earth hydroxide. Preferably about 1.0 eq. to about 2.0 eq., more preferably about 1.0 eq. to about 1.5 eq., and most preferably about 1.05 eq. to about 1.15 eq. of base with respect to compound (1) is used.

The reaction can be run under homogeneous conditions using an organic solvent or mixture of organic solvents. Examples for suitable organic solvents include: ketones such as acetone, 2-butanone and 4-methyl-2-pentanone, aromatic solvents such as toluene, halogenated solvents such as methylene chloride, ethers such as methyl tent-butyl ether, 2-methyltetrahydrofuran, and tetrahydrofuran, and esters such as ethyl acetate and isopropyl acetate.

Alternatively, the reaction can be conducted in a biphasic system including an aqueous phase and an organic phase. A biphasic system is advantageous compared to a homogeneous system because inorganic by-products can be removed by extraction. Any of the above mentioned organic solvents can be used as the organic phase as long as it is substantially immiscible with the aqueous phase.

The reaction can be conducted at a temperature in the range of about −50° C. to about +50° C., preferably about −10° C. to about 20° C.

The duration of the reaction is typically from about 30 min to about 90 min.

If a homogeneous system is employed the reaction mixture can be directly used for step (b) or optionally be washed with water or an aqueous basic solution prior to use in step (b). If a biphasic system is employed, the aqueous layer will be typically separated from the organic layer. The organic layer can then either be used as such or washed as indicated above before it is employed in step (b).

Alternatively, compound (3) can be isolated, e.g., by crystallization, before it is used in step (b).

The yield of step (a) is typically more than 90%.

In an alternative embodiment of step (a) a compound having the general formula (1 a) is reacted with a compound having the general formula (2) to form a compound having the general formula (3a)

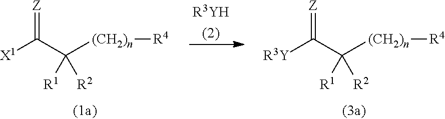

This embodiment is applicable if no cyclisation step (c) is to be conducted. In this case, the group R⁴ is not particularly limited and can be any group which does not negatively interfere with the reaction. Typically R⁴ is selected from the group consisting of hydrogen, optionally substituted alkyl, optionally substituted aryl, optionally substituted alkaryl, optionally substituted alkenyl, optionally substituted alkinyl, optionally substituted heteroaryl, optionally substituted heterocyclyl, and a leaving group X², wherein X² is as defined above. Preferably, R⁴ is X².

The above definitions of X¹, Z, R¹, R², n, R³, and Y are valid for this alternative embodiment.

The comments given above with respect to step (a) apply analogously to this alternative embodiment.

Step (b):

In step (b) the obtained compound having the general formula (3) is reacted with hydrazine to form a compound having the general formula (4)

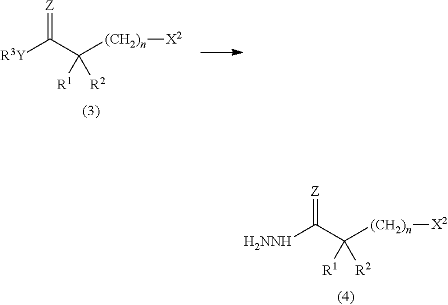

In the context of the present invention the term “hydrazine” is intended cover hydrazine as well as its salts and hydrazine hydrate. The type of salt is not particularly limited. Examples thereof include salts with inorganic acids such as hydrochloric acid, sulfuric acid, and phosphoric acid or organic acids such as acetic acid.

The present inventors have found that the reaction of chloropivalic acid chloride (compound (7)) with hydrazine gives high amounts of the corresponding diacylated compound (15) and only about 60% of the desired compound (10), even when hydrazine is used in a large excess (e.g., 10 eq.).

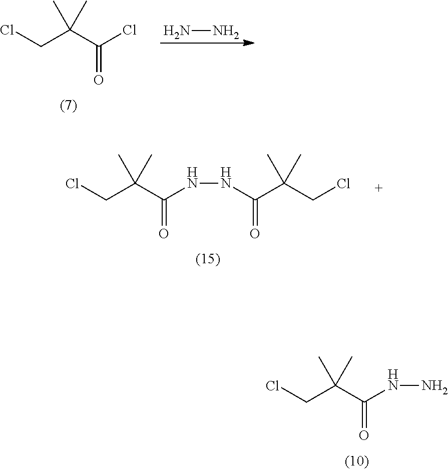

The present inventors found that the undesired diacylation leading to compound (15) can be suppressed by using the above defined acid compounds having the general formula (3) instead of acid chlorides as a starting material in step (b). For the step of the generation of the compounds having the general formula (3) the use of compounds having the general formula (2) above which have a pKa value of from about 1 to about 10, such as 2-mercaptobenzothiazole, leads to particularly favourable molar ratios of the compound of the general formula (4) to its diacylated congener. Preferably the reaction according to the present invention results in a molar ratio of the compound of the general formula (4) to its diacylated congener of at least about 3, more preferably at least about 5, even more preferably at least about 9.

In one embodiment of the invention, the solution obtained in step (a) or the isolated compound having the general formula (3) obtained in step (a) is reacted with aqueous hydrazine in the presence of an organic solvent to give the hydrazide having the general formula (4). Preferably, the solvent is the same as used in step (a). Most preferably, the organic solution containing the compound having the general formula (3) is directly added to an aqueous solution of hydrazine.

It is preferable to employ at least one equivalent of hydrazine in order to obtain a high conversion rate. In a preferred embodiment a slight excess of about 1.05 to about 1.50 equivalents of hydrazine compared to 1 equivalent of the compound having the general formula (3) is employed.

The reaction is typically conducted at a temperature in range of about −20° C. to about 80° C., preferably about −5° C. to about 15° C.

The duration of the reaction will depend on the chosen conditions and will usually be from about 30 min to about 120 min.

The yield of step (b) is usually about 90%.

The compound having the general formula (4) can be isolated according to procedures known in the art. However, in a preferred embodiment the reaction mixture obtained in step (b) is directly employed in step (c).

In an alternative embodiment of step (b) a compound having the general formula (3a) is reacted with hydrazine to form a compound having the general formula (4a)

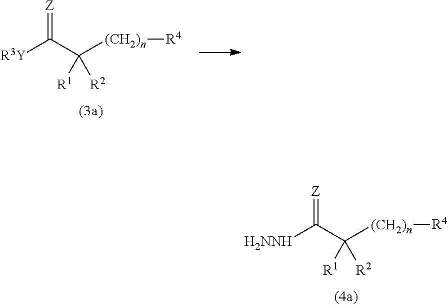

This embodiment is applicable if no cyclisation step (c) is to be conducted. In this case, the group R⁴ is not particularly limited.

The above definitions of R³, Y, Z, R¹, R², n and R⁴ are valid for this alternative embodiment.

The comments given above with respect to step (b) apply analogously to this alternative embodiment.

Step (c):

The compound having the general formula (4) forms a ring by intramolecular ring closure, which results in the compound having the general formula (5) in step (c) of the method of the present invention.

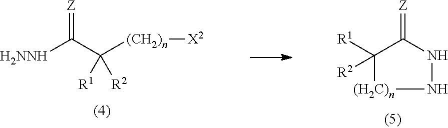

The conversion of the compound having the general formula (4) to the compound having the general formula (5) can be conducted by stirring in an organic solvent or in water or in mixtures thereof. The solvent is not particularly limited and is preferably the solvent employed in the previous step (b). Generally, steps (b) and (c) can be conducted in a one-pot reaction.

The temperature at which the reaction is conducted is not particularly limited and is usually from about 10° C. to about 100° C., preferably from about 30° C. to about 80° C.

The duration of the reaction will depend on the chosen temperature.

The reaction can be performed in the presence of an acid or base. However, the ring closure in absence of any additional acid or base is preferred.

In one embodiment, the aqueous solution of the compound having the general formula (5) as obtained in step (c) can be directly submitted to the hydrogenation reaction of step (d).

In an alternative embodiment if an organic solvent was employed, the compound having the general formula (5) can be extracted into an aqueous phase first, e.g. by addition of an aqueous acid such as hydrochloric acid. If X² is a halogen or a different anion of a strong acid, the addition of an aqueous acid may be omitted. This aqueous solution can be directly used for the next step. Any residual compound having the general formula (2) will either be contained in the organic phase or be present as a precipitate that can be removed by filtration.

In yet another embodiment the compound of the general formula (5) can be concentrated before it is submitted to step (d). In this case, the pH of the aqueous solution is preferably adjusted to be basic, e.g., more preferably to be in the range from about 5 to about 9, even more preferably from about 6 to about 8. Various bases can be used, which include ammonium, alkali or alkaline earth hydroxides (e.g., NH₄OH, NaOH, KOH, LiOH) or ammonium, alkali or alkaline earth carbonates (e.g., Na₂CO₃, K₂CO₃, or Li₂CO₃), or ammonium, alkali or alkaline earth hydrogencarbonates (e.g., NaHCO₃, KHCO₃, or LiHCO₃).

After pH adjustment the mixture can be concentrated, e.g., by evaporating the water under reduced pressure and/or by distillation. If desired, an organic solvent can be added in order to dissolve the compound of the general formula (5). Suitable organic solvents include, e.g., C₁₋₈ alcohols, such as 2-butanol, 1-butanol, 2-methyl-1-propanol, 2-propanol, ethanol, or methanol. Optionally, the solvent can be added prior to the concentration. Preferably the concentration steps are carried out in an inert atmosphere, for example under an oxygen-free atmosphere, such as nitrogen.

The concentration is preferably continued until the water content of the mixture of the compound having the general formula (5) and the organic solvent is below 10 wt.-%, more preferably below 1 wt.-%.

During concentration salts such as NaCl can precipitate, which can be removed, for example by filtration, prior to the hydrogenation step.

If filtration is performed, it should be conducted at a temperature where the product remains in solution and the salts are insoluble. Preferably, such a filtration is performed near or up to 15° C. below the boiling point of the solvent. If 2-propanol is employed as an organic solvent, then the filtration is preferably performed at 50° C. to 70° C. If 2-butanol or 2-methyl-1-propanol are employed as an organic solvent, then the filtration is preferably performed at 50° C. to 100° C.

The yield of the compound having the general formula (5) is typically more than 80% starting from the compound having the general formula (1) and using activated mercaptobenzothiazol as a reactant of the general formula (2).

Step (d):

The ring of the compound having the general formula (5) is opened in step (d) to form the desired ω-amino-alkane(thio)amide having the general formula (6).

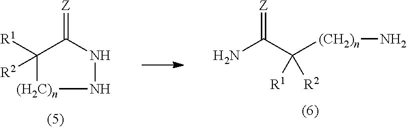

The reaction can be conducted under any conditions which are suitable for opening the ring.

In a preferred embodiment a reductive reaction can be used for the ring opening reaction. Examples of suitable reductive reactions include hydrogenation reactions and reductions employing complex hydrides (such as LiAlH₄ and NaBH₄), dissolving metal conditions (e.g., Na in NH₃) or electrochemical reductions. Examples of suitable hydrogenation reactions include hydrogenations using H₂ and transfer hydrogenations in the presence of a metal (such as transition metals), preferably hydrogenations using H₂ are employed. In a preferred embodiment, Raney nickel can be used as a hydrogenation catalyst because the hydrogenation can be conducted under normal pressure. However, elevated pressure can be employed, if a shorter reaction time is desired.

The hydrogenation conditions will depend on the type of hydrogenation reaction and can be determined by a person skilled in the field.

If hydrogenation using Raney nickel is chosen, the catalyst loading will be typically in the range of about 10 to about 200 wt.-% (based on compound (5)). In a preferred embodiment about 30 to about 100 wt.-% of catalyst (based on compound (5)) are used.

The hydrogenation is preferably conducted at about 10° C. to about 100° C., more preferably at about 40° C. to about 80° C. However, higher temperatures can be applied if the reaction is carried out under pressure from about 1 atm (101 kPa) to about 200 atm (20.3 MPa).

The hydrogenation is preferably conducted in an organic solvent. Suitable organic solvents include C₁₋₈ alcohols, such as 2-butanol, 2-methyl-1-propanol, 1-butanol, 2-propanol, ethanol, and methanol.

At about 60° C. and a H₂ pressure of about 1 atm (101 kPa) and using about 100 wt.-% Raney nickel catalyst (based on compound (5)) the reaction will be complete within about 2 h to about 20 h.

After the hydrogenation, the catalyst can be removed, e.g., by filtration and can be washed with the reaction solvent. If desired, the catalyst can be directly re-used.

If desired, the compound having the general formula (6) can be isolated and purified further by measures which are known in the art, such as crystallization and distillation.

The present invention provides a simple and convenient method for preparing an ω-amino-alkane(thio)amide having the general formula (6) which is suitable for production on an industrial scale. The method can provide the desired product in a high yield. The purity of the obtained ω-amino-alkane(thio)amide having the general formula (6) is high, so that it can be employed in the preparation of pharmaceuticals such as aliskiren. The method is particularly advantageous because no high pressure equipment is necessary and the reaction times are short. Since the intermediates of the general formula (3), (4) and (5) do not need to be isolated a further reduction of time and costs is achieved.

The following examples describe the present invention in detail, but they are not to be construed to be in any way limiting for the present invention.

### EXAMPLES

All examples were carried out under an atmosphere of nitrogen, if necessary.

### Example 1

In a 10 L reaction vessel equipped with a mechanical stirrer, a thermometer, and a pH probe 939 g of 2-mercaptobenzothiazole (5.5 mol, 1.1 eq.) were dissolved under nitrogen in 2700 mL of Me-THF at room temperature. After adding 290 mL of NaOH (50%) the mixture was stirred vigorously for 30 min. Then the temperature of the resulting biphasic suspension was adjusted to 0±2° C. Under vigorous stirring 652 mL of chloropivalic acid chloride (5.0 mol, 1.0 eq.) were added during 2 h at a rate that the bulk temperature was kept at 2±3° C. The resulting suspension was stirred for an additional 30 min before 500 mL of cold water (2±3° C.) were added to dissolve the precipitated NaCl. After stirring for 5 min the layers were separated and the aqueous layer was discarded.

The organic layer was cooled to 0±2° C. and added to 365 mL of cold (0±2° C.) aqueous hydrazine hydrate (80%, 60 mol, 1.2 eq.) placed in a 10 L reaction vessel (equipped with a mechanical stirrer, a thermometer, and a pH probe) at a rate that the bulk temperature was kept at 7±3° C.

After complete addition the bulk temperature was raised to 60±2° C. After stirring the mixture at this temperature for 1 h the pH was adjusted from pH 2.3 to pH 2.0±0.1 with 46 mL of 37% aqueous HCl. After stirring for 5 min the layers were separated. Then 500 mL of water were added to the organic layer and the pH was adjusted to pH 2.0±0.1 with −10 mL of 37% aqueous HCl. After stirring for 5 min the layers were separated. The combined aqueous layers were extracted twice with 170 mL of Me-THF (each). Then the aqueous layer was concentrated at a jacket temperature of 60±5° C. under reduced pressure (<100 mbar) to a mass of approx. 1130 g. This solution was transferred into a 2 L reaction vessel (equipped with a mechanical stirrer, a thermometer and a pH probe). The pH of the mixture was adjusted to pH 7.5±0.3 by addition of 115 mL of NaOH (50%) keeping the bulk temperature below 60° C. Then 1000 mL of 2-methyl-1-propanol were added and the biphasic mixture was heated to 60±5° C. Then—under vigorous stirring—the pH was re-adjusted to pH 7.5±0.3 with 10 mL of NaOH (50%). After stirring for 5 min at 60±5° C. 190 mL of water were added to dissolve the precipitated NaCl. After stirring for 5 min the layers were separated and the aqueous layer was transferred back into the reaction vessel and heated to 60±5° C. Then 500 mL of 2-methyl-1-propanol were added and at a bulk temperature of 60±5° C. 30 mL of water were added to dissolve the precipitated NaCl. After stirring for 5 min the layers were separated and the aqueous layer was transferred back into the reaction vessel and heated to 60±5° C. Then 500 mL of 2-methyl-1-propanol were added and at a bulk temperature of 60±5° C. 20 mL of water were added to dissolve the precipitated NaCl. After stirring for 5 min the layers were separated. The combined organic layers were concentrated at a bulk temperature of 60±5° C. under reduced pressure (<100 mbar) to a mass of approx. 1000 g. Then 200 mL of 2-methyl-1-propanol were added and the resulting solution was concentrated at a bulk temperature of 60±5° C. under reduced pressure (<100 mbar) to a mass of approx. 1000 g. Then 200 mL of 2-methyl-1-propanol were added and the resulting solution was concentrated at a bulk temperature of 60±5° C. under reduced pressure (<100 mbar) to a mass of approx. 1000 g. The resulting suspension was heated to 60±5° C. and the solids (NaCl) were filtered and washed with 50 mL of warm 2-methyl-1-propanol (50° C.). The combined filtrates were concentrated at a bulk temperature of 60±5° C. under reduced pressure (<100 mbar) to a mass of approx. 900 g.

In the meantime a 2 L reaction vessel was charged with 225 mL of Raney-Ni suspension (in water) and washed three times with 250 mL of MeOH (each) at room temperature and three times with 250 mL of 2-methyl-1-propanol (each) at 60±5° C. for 30 min. The warm (60±5° C.) 2-methyl-1-propanol solution from above was added into the vessel and the vessel was sealed, evacuated, and charged with nitrogen (1 bar, this procedure was repeated once). Then the vessel was evacuated and charged with hydrogen (1 bar, this procedure was repeated twice). Under vigorous stirring the suspension was heated to a bulk temperature of 60±5° C. After full conversion the vessel was evacuated and flushed with nitrogen (this procedure was repeated once). The catalyst was filtered and washed twice with 250 mL of warm (50±10° C.) 2-methyl-1-propanol (each). The combined 2-methyl-1-propanol fractions were concentrated at a bulk temperature of 60±5° C. under reduced pressure (<100 mbar) whereby approx. 320 g of product were obtained. This solution was cooled to 20±5° C. under stirring. To the resulting suspension 1000 mL of isobutyl acetate were added during 60 min. The resulting suspension was cooled to 0±2° C. and stirred for 2 h. The solid was collected by filtration, washed with 250 mL of isobutyl acetate, and dried at 40±5° C. under reduced pressure (<100 mbar) for 17 h to give 205 g of 3-amino-2,2-dimethylpropionamide (compound (12), 70% yield).

### Comparative Example 1

The following reaction was conducted using chloropivalic acid chloride as a starting material.

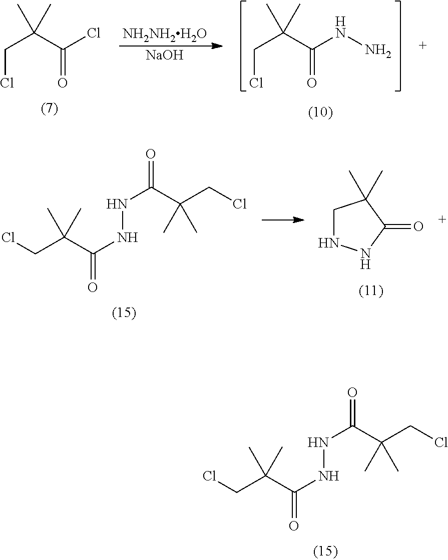

A 1L-reaction vessel equipped with a stirrer, thermometer and a dropping funnel was charged with 625 mL of H₂O. After cooling to 5° C., NaOH (20.2 g, 0.505 mol) was added, followed by NH₂NH₂.H₂O (43.8 g, 0.625 mol, 42.5 mL). The reaction mixture was then cooled to −2° C., and chloropivalic acid chloride (7) (77.5 g, 0.5 mol, 64.6 mL) was added within 90 min, keeping the reaction temperature between 0 and 5° C. After ⅔ of the addition the formation of white flakes was observed, indicating the formation of the diacylated product (15). After completion of the addition, stirring was continued for 30 minutes, then the reaction mixture was heated to 60° C. and stirred at this temperature for 1 h. Then the reaction mixture was cooled to ˜40° C. and the pH was adjusted to pH 2 by addition of concentrated HCl. HPLC analysis of the mixture showed an area % ratio of (11):(15)=1.5.

In a commercial process the diacylated product (15) would have to be separated from the desired product (11), which is time- and cost-intensive and would significantly reduce the overall yield of the process. Furthermore, it is to be expected that impurities of the diacylated product (15) would remain in the desired product (11). Therefore, the comparative process is not commercially viable.

The following HPLC-procedure was used:

Apparatus: HPLC Hewlett Packard HP-1100

Column: YMC Hydrosphere C18
HS12S03-1546WT; HS-302-3
No. 041591477(W)
150×4.6 mm I.D.
S-3 μm

Injection: 5 μL

Flow: 1.00 mL/min

Temperature: 40° C.

Stop-time: 6 min

Detection: 200 nm

Mobile phase:
Eluent A: 3.884 g sulfamic acid+1000 g water (HPLC grade)

Gradient: isocratic

Sample preparation: product analysis: Take approx. 15 mg solid; dilute in 25 mL eluent A.

Solvent: Eluent A

1. A method for the preparation of an w-amino-alkaneamide or an w-amino-alkanethioamide having the general formula (4), the method comprising the steps of:
(a) reacting a compound having the general formula (1) with a compound having the general formula (2) to form a compound having the general formula (3) wherein: 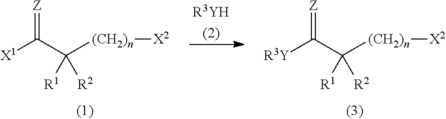
X¹ is selected from the group consisting of halogen and R—C(O)—O—, wherein R is a C₁₋₈ alkyl group;
X² is a leaving group selected from the group consisting of halogen;
—OSO₂R, wherein R is a C₁₋₄ alkyl group which is optionally substituted with one or more halogens or wherein R is a C₅₋₁₂ aryl which is optionally substituted with C₁₋₄ alkyl, NO, or CN;
Z is O or S;
R¹ and R² are independently selected from the group consisting of H and C₁₋₈ alkyl, wherein at most one of R¹ and R² is H;
n is an integer from 1 to 5;
R³ is selected such that the compound having the general formula (2) has a pKa value of at most about 11; and
Y is selected from the group consisting of O, NH and S; and
(b) subsequently reacting the compound having the general formula (3) with hydrazine to form a compound having the general formula (4) at a temperature in range of about −5° C. to about 15° C.

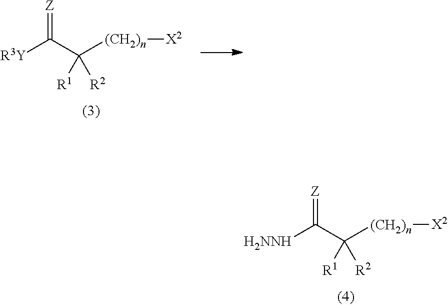

2. The method of claim 1, wherein R¹ and R² are CH₃ and n is 1.

3. The method of claim 1, wherein R³ is selected from the group consisting of substituted or unsubstituted aryl groups, substituted or unsubstituted heteroaryl groups, substituted or unsubstituted arylalkyl groups, substituted or unsubstituted heteroarylalkyl groups, substituted or unsubstituted arylacyl groups, and substituted or unsubstituted heteroarylacyl groups.

4. The method of claim 1, wherein the compound having the general formula (2) is the compound of the formula (8):

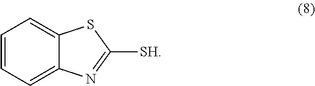

5. (canceled)

6. The method of claim 1, wherein step (a) is conducted in a biphasic system.

7-9. (canceled)

10. A compound having the general formula (13a) 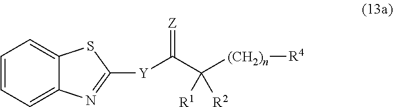
wherein
Y is selected from the group consisting of O, NH and S;
Z is O or S;
R¹ and R² are independently selected from the group consisting of H and C₁₋₈ alkyl,
wherein at most one of R¹ and R² is H;
n is an integer from 1 to 5; and
R⁴ is a leaving group selected from the group consisting of halogen;
—OSO₂R, wherein R is a C₁₋₄ alkyl group which is optionally substituted with one or more halogens or wherein R is a C₅₋₁₂ aryl which is optionally substituted with C₁₋₄ alkyl, NO, or CN.

11. (canceled)

12. The compound according to claim 10, wherein Y is S.

13. The compound according to claim 12, which has the general formula (9)

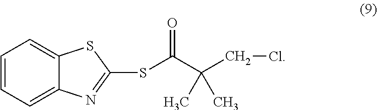

14. A compound having the general formula (4a) 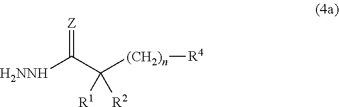
wherein
Z is O or S;
R¹ and R² are independently selected from the group consisting of H and C₁₋₈ alkyl,
wherein at most one of R¹ and R² is H,
n is an integer from 1 to 5;
R⁴ is a leaving group selected from the group consisting of halogen;
—OSO₂R, wherein R is a C₁₋₄ alkyl group which is optionally substituted with one or more halogens or wherein R is a C₅₋₁₂ aryl which is optionally substituted with C₁₋₄ alkyl, NO, or CN.

15. (canceled)

16. (canceled)

17. The method of claim 1, wherein the compound of formula (15a) 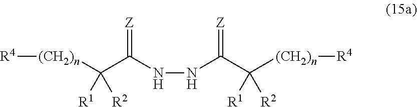
is formed in an amount such that the molar ratio of the compound of the general formula (4) to the compound (15a) is at least about 3.

18. The method of claim 1 further comprising the steps of
(a) cyclisation of the compound having the general formula (4) to form a compound having the general formula (5); 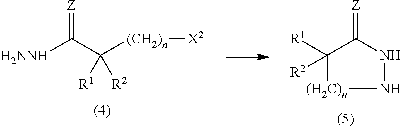
and
(b) opening the ring of the compound having the general formula (5) to form the w-amino-alkaneamide or an w-amino-alkanethioamide having the general formula (6).

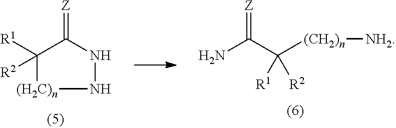

19. The method of claim 18 for the preparation of 3-amino-2,2-dimethylpropionamide having the formula (12), the method comprising the steps of:
(a) reacting a compound having the formula (7) with a compound having the formula (8) to form a compound having the formula (9); 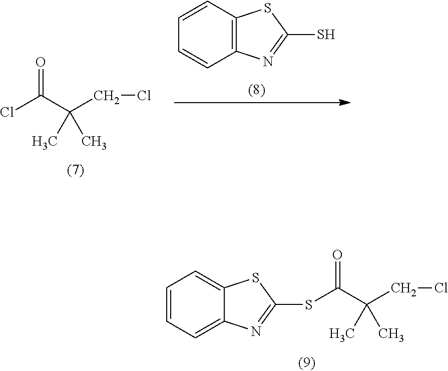
(b) subsequently reacting the compound having the formula (9) with hydrazine to form a compound having the formula (10); 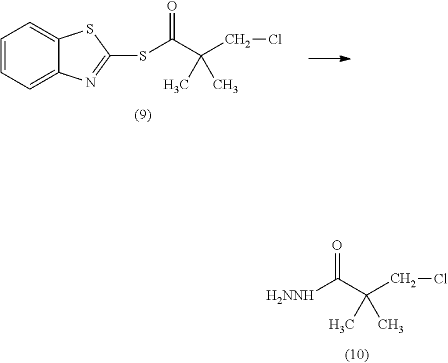
(c) cyclisation of the compound having the formula (10) to form a compound having the formula (11); 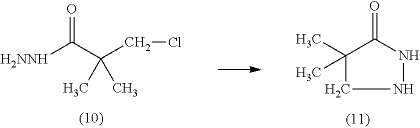
and
(d) opening the ring of the compound having the formula (11) to form 3-amino-2,2-dimethylpropionamide having the formula (12).

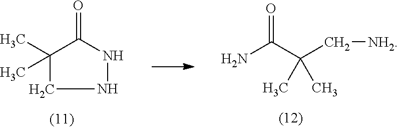

20. The method of claim 18, wherein step (b) is conducted by hydrogenation using Raney nickel.
